(19) United States
(12) Patent Application Publication
(10) Pub. No.: US 2021/0330903 A1
(43) Pub. Date: Oct. 28, 2021

(54) Multiple Dose Nasal Spray of Naloxone

(71) Applicant: Navinta III Inc, Boca Raton, FL (US)

(72) Inventor: Sandipkumar Arvindbhai Patel, Robbinsville, NJ (US)

(21) Appl. No.: 17/242,595

(22) Filed: Apr. 28, 2021

Related U.S. Application Data
(60) Provisional application No. 63/016,622, filed on Apr. 28, 2020.

(51) Int. Cl. A61M 15/00; A61M 15/08; A61K 31/485

(52) U.S. Cl. CPC A61M 15/0066; A61M 15/009; A61M 2202/04; A61M 15/0001; A61K 31/485; A61M 15/08

(57) ABSTRACT

A multiple dose Naloxone spray dosage form provides a container having two or more unit doses of Naloxone or a salt thereof in a liquid that is delivered by a metered dosage spray pump. In different embodiments, ten or more, 3-500, and 10-500 unit doses are provided in the container. The metered dosage spray pump delivers 50 to about 200 μL of Naloxone solution per spray, preferably, 100 μL per spray. Each unit dose contains about 0.5 mg to 12 mg of Naloxone or a pharmaceutically acceptable salt thereof, preferably, about 2 mg to 4 mg thereof in the delivered spray. A stable pharmaceutically acceptable composition of Naloxone is used in the multiple dose Naloxone spray dosage form. A method of treatment of opioid overdose involves administration of multiple doses using the multiple dose Naloxone spray dosage form.

### FIELD OF THE INVENTION

The present invention relates to multiple dose liquid formulations of Naloxone for nasal delivery.

### BACKGROUND OF THE INVENTION

The Centers for Disease Control and Prevention (CDC) issued an alert in October 2015 reporting an increase in fentanyl-related accidental overdose deaths due to illicitly manufactured fentanyl. Additionally, in August 2016, the CDC updated their previous alert after a sharp increase in the availability of counterfeit opioid and benzodiazepine pills being sold on the street containing varying amounts of fentanyl and other similar compounds. As a result, it is increasingly common for multiple overdoses of opioids within a short time period. There is an increasing need for treatments to reverse respiratory depression from opioid use.

Naloxone is indicated for the treatment of opioid toxicity, specifically to reverse respiratory depression from opioid use. It is useful in accidental or intentional overdose and acute or chronic toxicity. Common opioid overdoses treated with naloxone include heroin, fentanyl, carfentanil, hydrocodone, oxycodone, methadone, and others.

Naloxone hydrochloride is a white to slightly off-white powder, and is soluble in water, in dilute acids, and in strong alkali; slightly soluble in alcohol; practically insoluble in ether and in chloroform. Naloxone hydrochloride is chemically 17-Allyl-4,5α-epoxy-3,14 dihydroxymorphinan-6-one hydrochloride with the following structure:

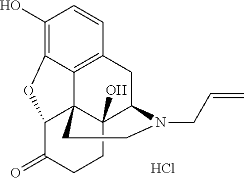 [SMILES: C=CCN1CC[C@]23c4c5ccc(O)c4O[C@H]2C(=O)CC[C@@]3(O)[C@H]1C5 ; Cl]

Naloxone hydrochloride antagonizes opioid effects by competing for the same receptor sites. Naloxone hydrochloride reverses the effects of opioids, including respiratory depression, sedation, and hypotension. It can also reverse the psychotomimetic and dysphoric effects of agonist-antagonists such as pentazocine.

Naloxone hydrochloride injection, 0.4 mg/mL and 1 mg/mL is approved by the United States Food and Drug Administration and is indicated for the complete or partial reversal of opioid depression, including respiratory depression, induced by natural and synthetic opioids including propoxyphene, methadone, and certain mixed agonist-antagonist analgesics: nalbuphine, pentazocine, butorphanol and cyclazocine. Naloxone hydrochloride is also indicated for the diagnosis of suspected or known acute opioid overdosage. Naloxone may be useful as an adjunctive agent to increase blood pressure in the management of septic shock.

Naloxone hydrochloride Injection, USP is a sterile, nonpyrogenic solution of naloxone hydrochloride in water for injection. Each milliliter (mL) contains 0.4 mg naloxone hydrochloride and sodium chloride to adjust tonicity in water for injection. It may contain hydrochloric acid for pH adjustment; pH 4.0 (3.0 to 6.5).

The single-dose solution contains no bacteriostat, antimicrobial agent or added buffer (except for pH adjustment) and is intended for use only as a single-dose injection. When smaller doses are required, the unused portion should be discarded.

A multiple-dose injection solution additionally contains 1.8 mg/mL methylparaben and 0.2 mg/mL propylparaben added as preservatives. The 0.4 mg/mL vial is also available in an unpreserved, paraben-free formulation containing 9 mg/mL of sodium chloride. Naloxone Hydrochloride Injection, USP may be administered intravenously, intramuscularly, or subcutaneously.

Injection products are difficult to administer and typically only used by trained medical professionals. Therefore, alternative dosage forms are desired.

NARCAN® (Naloxone hydrochloride) nasal spray is currently approved by the United States Food and Drug Administration and indicated for the emergency treatment of known or suspected opioid overdose, as manifested by respiratory and/or central nervous system depression. NARCAN® Nasal Spray is intended for immediate administration as an emergency therapy in settings where opioids may be present.

Each NARCAN® Nasal Spray contains a 2 mg or 4 mg single dose of naloxone hydrochloride (equivalent to 1.8 mg or 3.6 mg of naloxone) in a 0.1 mL (100 microliter) aqueous solution. Inactive ingredients include benzalkonium chloride (preservative), disodium ethylenediaminetetraacetate (stabilizer), sodium chloride, hydrochloric acid to adjust pH, and purified water. The pH range is 3.5 to 5.5.

NARCAN® nasal spray 2 mg is currently supplied as a carton containing four (4) blister packages (NDC 69547-212-04) each with a single spray unit and as a carton containing twenty-four (24) blister packages (NDC 69547-212-24) each with a single spray unit. NARCAN® Nasal Spray 4 mg is supplied as Carton containing two (2) blister packages (NDC 69547-353-02) each with a single spray unit.

Storage of Naloxone nasal spray is at room temperature 68° F. to 77° F. (20° C. to 25° C.) and the product must be protected from light. Excursions are permitted between 41° F. to 104° F. (5° C. to 40° C.). Naloxone hydrochloride nasal spray freezes at temperatures below 5° F. (−15° C.). If this happens, the device will not spray. It may be thawed by allowing it to sit at room temperature for 15 minutes, and it may still be used if it has been thawed after being previously frozen.

The prescribing label of NARCAN® Nasal Spray indicates that it is for intranasal use only. The instructions state, “Do not attempt to reuse NARCAN Nasal Spray.” When multiple doses are required, they must be re-administered using a new nasal spray product, every 2 to 3 minutes if the patient does not respond or responds and then relapses into respiratory depression.

When using a new NARCAN® Nasal Spray to give another dose, the nostril for dosing should be alternated. As the patient has to be given Naloxone spray every 2-3 minutes in each nostril the single dose nasal spray requires the person administering the dosage to continuously obtain, open blister pack packaging, remove a new single spray unit, and administer it. It is possible that the person administering the dosage may not be able to administer the dosages as quickly as needed and/or may be unable to take other actions such as call 911 for emergency help due to the time involved in administration of the NARCAN® single spray unit.

Further, disposal of multiple single use spray containers poses a higher risk to third parties such as children for exposure to the active ingredient.

There are many generic Naloxone Hydrochloride products approved by FDA as injection products. However, only one generic metered nasal spray (TEVA PHARMS USA) has been approved and that product is listed as “Discontinued”. Thus, there are presently no generic Naloxone Hydrochloride nasal sprays available to consumers in the U.S., although the nasal spray products are easier and safer to administer and easier for susceptible populations to keep on hand.

Therefore, there is an unmet need for generic alternatives to NARCAN® Nasal Spray, i.e., there is a need for commercially viable Naloxone Hydrochloride metered nasal products.

There are alternative (unapproved) nasal spray formulations to NARCAN disclosed in the literature.

For instance, U.S. Pat. No. 9,211,253 assigned to Opiant Pharmaceuticals Inc teaches a single-use, pre-primed device adapted for nasal delivery of a pharmaceutical composition to a patient by one actuation of said device into one nostril of said patient The device has a single reservoir comprising a pharmaceutical composition, which is an aqueous solution of about 100 μL comprising: about 4 mg naloxone hydrochloride or a hydrate thereof; between about 0.2 mg and about 1.2 mg of an isotonicity agent; between about 0.005 mg and about 0.015 mg of a preservative; about 0.2 mg of a stabilizing agent; an amount of an acid sufficient to achieve a pH or 3.5-5.5. However, the patent does not teach anything about multiple dose formulations.

U.S. Pat. No. 9,468,747 teaches a method of treatment of opioid overdose or a symptom thereof, comprising nasally administering to a patient in need thereof a dose of naloxone hydrochloride using a single-use, pre-primed device adapted for nasal delivery of a pharmaceutical composition to a patient by one actuation of said device into one nostril of said patient, having a single reservoir comprising a pharmaceutical composition which is an aqueous solution of about 100 μL. However, this patent also does not disclose a multiple dose naloxone formulation for nasal delivery containing more than 100 μL volume in a reservoir. Moreover, excipients are disclosed but are not indicated to be suitable for a multiple dose formulation.

U.S. Pat. Nos. 9,561,177; 9,775,838 and 10,085,937 teach methods of treating opioid overdose comprising: delivering a 25-200 μL spray of a pharmaceutical solution from a pre-primed device into a nostril of a patient, wherein the device is adapted for nasal delivery, and wherein the pharmaceutical solution comprises about 4 mg and 10 mg naloxone hydrochloride or a hydrate thereof, between about 0.005% and about 0.015% (w/v) of benzalkonium chloride, and an isotonicity agent. They also disclose a mist delivered from a pre-primed device, wherein the mist comprises droplets, wherein the droplets comprise, in aggregate, about 4 mg of naloxone hydrochloride or a hydrate thereof, between about 0.005% and about 1% (w/v) of benzalkonium chloride, and an isotonicity agent, wherein no more than about 10% of the droplets have a diameter less than 10 μm. Neither discloses a method of treating opioid overdose using multiple dose nasal delivery of Naloxone.

U.S. Pat. No. 4,464,378 describes a method for eliciting an analgesic or narcotic antagonist response in a warm-blooded animal, which comprises administering intranasally (IN) to said animal to elicit a narcotic antagonist response, a narcotic antagonist effective amount of naloxone.

WO 82/03768 discloses a composition that contains 1 mg of naloxone hydrochloride per 0.1 ml of solution adapted for nasal administration used in the treatment of narcotic induced respiratory depression (overdose) at a dosage approximately the same as that employed for intravenous (IV), intramuscular (IM) or subcutaneous (SQ) administration.

It is generally believed that due to the priming procedure and limited control of dosing, multi dose devices are less suited for drugs with a narrow therapeutic window. For expensive drugs intended for single administration or sporadic use and where tight control of the dose and formulation is of particular importance, single-dose or duo-dose spray devices are recommended. However, a multiple-dose Naloxone spray would require significantly less space for storage, making it convenient to carry and handy for use rather than carrying multiple number of single dose units. Commercial manufacturing of a multiple-dose formulation would be faster and easier because fewer number of units would need to be manufactured, labeled, packed, stored and shipped.

WO 00/62757 to Britannia Pharmaceuticals Ltd. teaches pharmaceutical compositions for intranasal or oral (PO) administration which comprise an opioid antagonist, such as naloxone, for application by spray in the reversal of opioid depression for treatment of patients suffering from opioid over-dosage, wherein the spray applicator is capable of delivering single or multiple doses and suitable dosage units are in the range of 0.2 to 5 mg. However, this application has lapsed in all jurisdictions and no such type of product has been approved by any regulatory agency, suggesting that the disclosed formulations are not viable.

There is a need for commercially viable Naloxone nasal spray dosage forms. Particularly, there is a need for Naloxone nasal spray dosage forms that are cost effective to manufacture and that are stable at commercial transportation and storage conditions.

There is an unmet need for a Naloxone spray dosage form that would allow for multiple administrations without having to unpack a new spray unit each time a dose is administered.

There is an unmet need for multiple-dose Naloxone dosage form for nasal delivery for treatment of opioid overdose and, particularly, those that are cost effective to manufacture and that are stable at commercial transportation and storage conditions.

Moreover, there is an unmet need for a multiple use nasal spray of Naloxone suitable to readily deliver Naloxone doses in rapid succession, which would allow the administrator more time to get medical emergency help.

There is further an unmet need for a multiple dose nasal Naloxone dosage form to ensure that overdose patients have enough doses of Naloxone on hand.

There is an unmet need for treating patients with opioid overdose using a multiple dose Naloxone nasal spray.

There is an unmet need for a multiple dose Naloxone nasal spray that is convenient to carry.

There is an unmet need for a multiple dose Naloxone nasal spray that is convenient to administer to a patient.

There is an unmet need for a multiple dose Naloxone nasal spray that allows an administration to get faster medical emergency help for a patient.

There is an unmet need for a multiple dose Naloxone nasal spray that is safe and easy to dispose of.

There is an unmet need for a multiple dose Naloxone nasal spray that is fast and easy to manufacture, label, store and ship.

It is an objective of the invention to provide Naloxone nasal spray formulations and metered dose Naloxone spray dosage forms that are suitable for multi-dose administration, are cost effective to manufacture, and that are stable at commercial transportation and storage conditions.

### SUMMARY OF INVENTION

The foregoing is achieved by the present invention, which discloses a method of treating opioid overdose patient using a multiple dose nasal spray dosage form of Naloxone or a pharmaceutically acceptable salt thereof.

The invention provides a multidose device for nasal delivery of Naloxone or its pharmaceutically acceptable salt thereof.

The invention provides formulation compositions of Naloxone or a pharmaceutically acceptable salt thereof suitable for a multiple dose spray dispenser, such as a metered dose spray dispenser.

A stable pharmaceutically acceptable composition of Naloxone suitable for a multidose dispenser comprises Naloxone or a pharmaceutically acceptable salt thereof, and an aqueous intranasal carrier. The composition is stable for at least 6 months when stored at room temperature in a container having a metered dosage spray pump and actuator mounted to the container.

In preferred embodiments, the composition comprises one or more of a tonicity adjusting agent, pH adjusting, buffering agent, antioxidant, permeation enhancer, chelating agent, preservative, solvent, sugar, and gelling agent.

In certain preferred embodiments, the composition comprises a preservative, which is preferably phenyl ethyl alcohol.

In some preferred embodiments, the composition comprises permeation enhancer. The permeation enhancer is preferably selected from disodium edetate and caffeine.

A multiple dose device containing Naloxone delivers more than 2 doses, preferably 3 to 100 doses, more preferably 3 to 50 doses.

In certain embodiments, a multiple dose Naloxone dosage form comprises more than 10 mg Naloxone or its pharmaceutically acceptable salt, preferably more than 50 mg, more preferably more than 100 mg.

In certain embodiments, a Naloxone formulation suitable for a multiple dose spray dispenser comprises one more pharmaceutically acceptable excipients, such as but not limited to pH adjusting agents, tonicity adjusting agents, anti-oxidants, preservatives, sugars, organic solvents, co-solvents, chelating agents, alcohols, acids, salts, esters, buffers or combination thereof.

In certain embodiments, the formulation comprises in-situ gel formation properties and muco-adhesion properties.

In some embodiments, the multiple dose device is used to deliver Naloxone or its pharmaceutically acceptable salt to patient in a lying position through a nasal cavity.

In certain embodiments, the multiple dose device can deliver the Naloxone or a pharmaceutically acceptable salt thereof through a nasal cavity in less than 30 seconds, more preferably less than 20 seconds, most preferably less than 10 seconds.

In some embodiments, the multiple dose nasal spray delivers about 50 to 200 μL volume containing about 2 to 10 mg of Naloxone in each spray.

In certain embodiments, the formulation is a Naloxone hydrochloride nasal spray solution that freezes below −15° C. to below about −25° C.

In some embodiment, the osmolality of the Naloxone solution is about 200 mOsmol/L to about 800 mOsmol/L.

In certain embodiments, the multiple dose device is made up of at least a pump delivery product having a reservoir filled with Naloxone solution. The reservoir contains at least 1 mL to 100 mL Naloxone product solution, more preferably 2 mL to 50 mL Naloxone solution. In preferred embodiments, the concentration of Naloxone in the solution is about 10 mg/mL to 200 mg/mL.

In certain embodiments, the pump is made up of polymeric material, non-polymeric material, or a combination thereof.

In some embodiments of the invention, Naloxone or its pharmaceutically acceptable salt can be administered along with one or more other active ingredients, such as, but not limited to, Buprenorphine, Pentazocine, Morphine, and Amifostine.

A method of manufacturing a multiple dose nasal spray of Naloxone or a pharmaceutically acceptable salt thereof is also within the scope of the invention.

A method of manufacturing a pharmaceutically acceptable multi-dose intranasal Naloxone product comprises the steps of solubilizing intranasally acceptable excipients in water to form an intranasal carrier; solubilizing Naloxone or a pharmaceutically acceptable salt thereof in the intranasal carrier to form a Naloxone solution; adding an amount of acid sufficient to adjust pH of the Naloxone solution to about 3 to about 6; and filling two or more unit doses of the Naloxone solution into a container of a multiple dose nasal spray device.

In some embodiments of the method, the filling step comprises filling 10 to 500 unit doses of the of the Naloxone solution into the container.

A method of treating opioid overdose or toxicity in a patient comprises intranasally administering to the patient multiple doses of the Naloxone composition in a liquid carrier contained in a multiple dose spray dispenser containing two or more unit doses of the Naloxone in a liquid carrier.

The multiple dose spray dispenser is placed in a first nostril of the patient, and the spray dispenser is actuated to spray the Naloxone in a liquid carrier in the first nostril; the multiple dose spray dispenser is placed in a second nostril of the patient, and the spray dispenser is actuated to spray the Naloxone in a liquid carrier in the second nostril. In certain embodiments, the steps are repeated using same multiple dose spray dispenser every two to three minutes as needed.

In preferred embodiments, each dose contains about 0.5 mg to 6 mg of Naloxone or a pharmaceutically acceptable salt thereof.

### BRIEF DESCRIPTION OF THE DRAWINGS

The foregoing summary, as well as the following detailed description of the embodiments, will be better understood when read in conjunction with the appended figures. For the purpose of illustration, the figures may describe the use of specific embodiments. It should be understood, however, that the formulations described herein cannot be limited to the precise embodiments discussed or described in the figures.

FIG. 1 shows a partial cross-section and elevation view of a multiple dose Naloxone spray dispenser containing a formulation of the present invention.

FIG. 2 shows a perspective view of a bottle and metered dose pump device containing a formulation of the present invention.

FIG. 3 is a table showing the stability of Composition 1 of Example 1 held in a multiple dose Naloxone spray dispenser at 25° C. and 40° C. and tested after storage time periods of 1, 2, 3 and 6 months.

FIG. 4 is a table showing the stability of Composition 2 of Example 1 held in a multiple dose Naloxone spray dispenser at 25° C. and 40° C. and tested after storage time periods of 1, 2, 3 and 6 months.

FIG. 5 is a table showing the stability of Composition 3 of Example 1 held in a multiple dose Naloxone spray dispenser at 25° C. and 40° C. and tested after storage time periods of 1, 2, 3 and 6 months.

FIG. 6 is a table showing the stability of Composition 4 of Example 1 held in a multiple dose Naloxone spray dispenser at 25° C. and 40° C. and tested after storage time periods of 1, 2, 3 and 6 months.

### DETAILED DESCRIPTION

The present invention discloses a method of treating opioid overdose patients by using multiple dose Naloxone spray by nasal route of administration.

The method involves administering a composition comprising Naloxone or a pharmaceutically acceptable salt thereof. The composition is delivered via nasal route of administration.

Naloxone as used herein refers to N-allylnoroxymorphone or as 17-allyl-4,5α-epoxy-3,14-dihydroxymorphinan-6-one having the structure shown in Formula I:

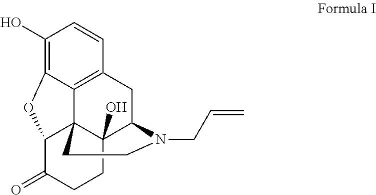 [SMILES: C=CCN1CC[C@]23c4c5ccc(O)c4O[C@H]2C(=O)CC[C@@]3(O)[C@H]1C5]

Naloxone is a racemic mixture of two enantiomers, (−)-naloxone (levonaloxone) and (+)-naloxone (dextronaloxone), only the former of which is active at opioid receptors. The invention includes mixtures of both enantiomers.

Pharmaceutically acceptable salts of Naloxone include but not limited to acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic acid salt.

An especially preferred salt is Naloxone hydrochloride.

Typically, more than 10 mg Naloxone or a salt thereof, preferably more than 50 mg, more preferably more than 100 mg will be placed in a spray dispenser. About 0.5 mg to 12 mg of Naloxone or a pharmaceutically acceptable salt thereof are administered per unit dose; but more preferably, about 2 mg to 10 mg of Naloxone are administered per unit dose.

The Naloxone is dissolved in a solvent. Some of the preferred solvents for use in liquid formulations of the present invention are, but not limited to, alcohols, such as ethanol or isopropanol, low molecular weight fatty acids, propylene glycol, polyethylene glycol, glycerin, water, and mixtures thereof.

The concentration of Naloxone in the liquid formulations herein ranges from 10 mg/mL to 200 mg/mL, more preferably 10 mg/mL to 60 mg/m L.

The compositions may also include one or more of buffers, pH adjusting agent, chelating agent, tonicity agent, antioxidant,

Buffers suitable for use in the pharmaceutical compositions described herein include, but are not limited to, pharmaceutically acceptable salts and acids of acetate, glutamate, citrate, tartrate, benzoate, lactate, histidine or other amino acids, gluconate, phosphate, malate, succinate, formate, propionate, and carbonate.

Suitable pH adjusting agents which may be used in the present invention include, but not limited to sodium hydroxide, hydrochloric acid, citric acid, acetic acid, fumaric acid, hydrochloric acid, malic acid, nitric acid, phosphoric acid, propionic acid, sulfuric acid, tartaric acid, or combinations thereof. In certain preferable embodiments, the formulations comprise a pH adjusting agent. In some of those embodiments, the pH adjusting agent is sodium hydroxide and/or hydrochloric acid.

The compositions of Naloxone are stable.

As used herein to describe the formulations of the present invention the term “stable” means a change of not more than about 10% in the concentration of the active agents when measured by HPLC.

The formulations may optionally comprise chelating agent at about 0.01% to 5.0% w/w of the formulation, preferably about 0.1% to 2.0% w/w. Suitable chelating agents which may be used in the present invention include, but not limited to edetate disodium (EDTA); edetate trisodium, edetate tetrasodium; and diethylene amine pentaacetate or derivatives thereof. In certain preferable embodiments, the formulations comprise disodium edetate.

The formulations may also comprise permeation enhancer at about 0.01% to 10.0% w/w of the formulation, preferably about 0.1% to 5% w/w. Suitable permeation enhancers which may be used in the present invention include, but are not limited to phenylethyl alcohol, disodium edetate, N-methyl-pyrrolidone, salcaprozate sodium, salcaprozic acid, polysorbate 80, sodium lauryl sulfate, caffeine, and combinations thereof.

Viscosity enhancing agent may help to retain the product solution on nasal mucosa for longer time. These agents may also increase viscosity after administration to the nostril, such as through in-situ gel formation and/or muco-adhesion, to promote retention of the drug formulation on the nasal mucosa.

The formulation may optionally comprise viscosity enhancing agent at about 0.01% to 5% w/w of the formulation, preferably about 0.1% to 2.5% w/w. Suitable viscosity enhancing agents which may be used in the present invention include, but are not limited to, chitosan, pectin, poloxamer, carbopol, polyacrylates, hypromellose, xanthan gum, starch, xyloglucan, and combinations thereof.

The formulations of the present invention may further comprise a tonicity adjusting agent at 0.2% to 10% w/v of the formulation, preferably 0.5% to 5% w/v. Tonicity adjusting agents suitable for use in pharmaceutical compositions described herein include, but are not limited to, anhydrous or hydrous forms of sodium chloride, dextrose, sucrose, xylitol, fructose, glycerol, sorbitol, mannitol, potassium chloride, mannose, and other inorganic salts except those containing divalent cations. In certain preferable embodiments, the formulations comprise sodium chloride.

Antioxidants that may be included are, for example, alkyl gallates, butylated hydroxy anisole (BHA) butylated hydroxytoluene (BHT), nordihydroguaiaretic acid, alpha-tocopherol, ascorbic acid and sodium metabisulfite (especially ascorbic acid).

Suitable antioxidants include butylated hydroxy anisole (BHA) butylated hydroxytoluene (BHT), alpha-tocopherol, and ascorbic acid, particularly alpha-tocopherol and ascorbic acid, particularly alpha-tocopherol and ascorbic acid, and most particularly ascorbic acid. Ascorbic acid is commonly referred to as Vitamin C. Alpha-tocopherol is commonly referred to as Vitamin E.

In some embodiments, formulations of the invention will contain one or more (e.g., one or two) antioxidants. Normally, one antioxidant is suitable. When the formulation comprises more than one antioxidant, the formulation suitably comprises two antioxidants selected from alkyl gallates, butylated hydroxy anisole (BHA) butylated hydroxytoluene (BHT), nordihydroguaiaretic acid, alpha-tocopherol, ascorbic acid and sodium is metabisulfite. Most preferably, the two antioxidants are alpha-tocopherol and ascorbic acid.

In other embodiments, suitable antioxidants for inclusion include those which are pharmaceutically acceptable for use in human and veterinary formulations, although not limited to those currently regarded as safe by any regulatory authority. For example, the antioxidant can be selected from among lipoic acid, thioglycerol (also known as monothioglycerol) and analogs thereof, propyl gallate, methionine, cysteine, metabisulfites, sodium formaldehyde sulfoxylate, phenol-containing aromatic and aliphatic compounds, dihydrolipoic acid, and mixtures of the foregoing.

Preservatives as used in the present compositions may be selected from one or more of benzalkonium chloride, benzyl alcohol, chlorobutanol, cresol, ethyl alcohol, 2-phenylethanol, thiomersal, parabens (alkylparabens), benzoic acid, EDTA, sodium benzoate and the like thereof.

In certain embodiments, the invention includes a method of manufacturing a multiple dose naloxone nasal spray. Formulation of the compositions will generally involve solubilizing excipients in water, and solubilizing the Naloxone active ingredient. The formulations are placed in a suitable multidose spray dispenser.

The number of doses per device is typically 3 to 500. Each unit dose metered by the metered dosage spray pump contains about 50 μL to about 200 μL of Naloxone or a pharmaceutically acceptable salt thereof in a liquid nasal carrier.

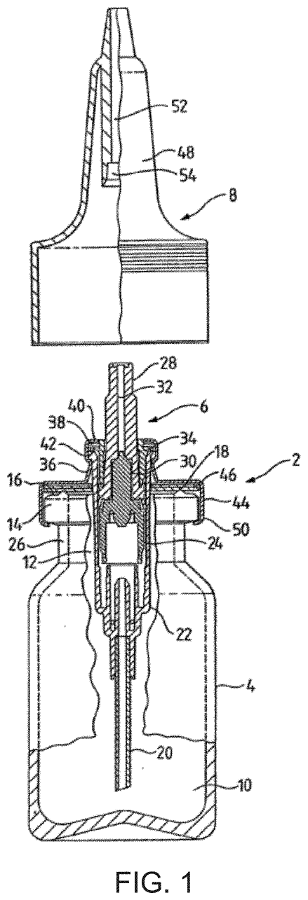

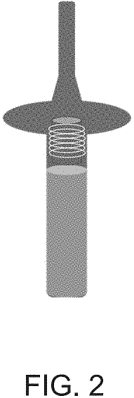

A suitable multidose spray dispenser has a pump and reservoir and the reservoir contains about 1 to 50 mL of the liquid Naloxone composition. Exemplary devices suitable for the invention include those containing a reservoir (e.g., a bottle), pump and actuator, such as those shown in FIGS. 1 and 2.

These devices are held between the forefinger and middle finger with the thumb on the actuator. A push with the thumb actuates the dispenser and a metered dose is delivered. In contrast to a single dose device, metered-dose spray pumps require priming and some degree of overfill to maintain dose conformity for the labeled number of doses.

With reference to FIG. 1, an exemplary multiple dose spray dispenser 2 comprises a bottle 4, a pump unit 6, a pump actuator 8 and a cap (not shown) which fits over the pump actuator 8. A nasal preparation 10 is contained within the bottle 4. The preparation 10 consists of about 1 to about 50 mL of the Naloxone compositions described herein.

The bottle 4 can be made from a colored glass so as to protect the Naloxone composition 10 from light, and has a mouth 12 for receiving the pump unit 6. Alternatively, the bottle is made from HDPE. A mounting flange 14 for the pump unit 6 is provided around the mouth 12. The upper surface 16 of the flange is provided with a generally triangular sectioned rib 18.

The pump unit 6 provides a metered unit dosage with each pump of the actuator 8. Pump unit 6 can be of a type available commercially, such as VP7 from Valois Division Pharmacie, and comprises a dip tube 20 which extends down into the preparation 10 within the bottle 4, a main body 22 of plastics such as polypropylene, a valve 24 mounted within the body 22 a spring 26 and a stem. The stem 28 has a lower bore portion 30 which receives the valve 24, and a dispensing bore 32 extending therethrough. The stem 28 and valve 24 are slidable within the body 22, and a stop member 34 limits the upward movement of the stem 28. Each unit dose metered by the pump unit 6 contains about 50 μL to about 200 μL of Naloxone or a pharmaceutically acceptable salt thereof in the liquid nasal carrier. In some embodiments, each unit dose metered by the pump unit 6 contains about 100 μL of Naloxone or a pharmaceutically acceptable salt thereof in a liquid nasal carrier. Each unit dose contains about 0.5 mg to 12 mg of Naloxone or a pharmaceutically acceptable salt thereof. In some embodiments, each unit dose contains about 2 mg to 4 mg of Naloxone or a pharmaceutically acceptable salt thereof in the delivered spray.

The body 20 and the stop 34 are held together by a deformable aluminum collar 36. A gasket 38 is provided between the upper flange 40 of the collar 36 and an opposing flange 42 of the stop member 34.

The collar 36 also mounts the pump unit 6 on the bottle 4. To this end, the collar 36 also comprises a lower portion 44 which extends down and is deformed around the flange 14 of the bottle 4. A gasket 46 is positioned between the lower portion 44 of the collar 36 and the upper surface of the bottle flange 12. The annular rib 18 provided around the bottle mouth 12 engages with the gasket 46 to ensure an airtight joint between the collar 38 and the bottle 4.

The actuator 8 comprises a body 48 which is received over the outer surface 50 of the pump unit 6. The body 48 is provided with an internal bore 52 having an enlarged lower end 54 which fits over and engages the upper end of the stem 30.

The cap (not shown) will have suitable means for retaining the cap on the actuator 8.

To use the spray, a user inserts the actuator 8 into a nostril and presses the actuator 8 and bottle 4 toward each other. This actuates the pump unit 6 to dispense a predetermined dose of the preparation 10 into the user's nostril.

It will be appreciated that the collar mounting arrangement disclosed reduces the seepage of air into the bottle 4 when the spray is not being used, thereby improving the shelf life of the preparation 10.

Other dispensers, actuators and pump units suitable for delivering multiple metered doses of the formulations described herein are described in, e.g., U.S. Pat. Nos. 8,365,961; 9,151,281; 9,592,934; 10,160,577; and U.S. Pat. No. 10,202,224 to Nemera la Verpilliere SAS.

FIG. 2 shows an alternative bottle 104 that may be used with any suitable pump 116 and actuator 118. A nasal preparation 110 is contained within the bottle 114. The preparation 110 consists of a Naloxone composition described herein. The bottle 104 is preferably HDPE and has a mouth 114 and threaded neck 112 for receiving a pump unit that can be screwed on the threaded neck.

It is preferable that any nasal device is child resistant, such as a device with some type of lock that needs to be altered before actuation or device that require significant high level of external force to spray. Exemplary devices are Nemera SPRAY LOCK™ systems. Preferably, the device will be compliant with 16 CFR 1700.20 (2021).

A method of treating opioid overdose or toxicity in a patient comprises intranasally administering to the patient multiple doses of Naloxone in a liquid carrier contained in a multiple dose spray dispenser containing two or more unit doses of the Naloxone in a liquid carrier. The method includes placing an overdose patient in lying condition, placing the device in a first nostril of the patient, pressing the actuator to spray the Naloxone solution in the first nostril, placing the device in a second nostril, pressing the plunger to spray the Naloxone solution in the second nostril, repeating the procedure using same device every two-three minutes until the person responds or the emergency medical help is received.

### EXAMPLES

The formulations and methods described herein are now further detailed with reference to the following examples. These examples are provided for the purpose of illustration only and the embodiments described herein should in no way be construed as being limited to these examples. Rather, the embodiments should be construed to encompass any and all variations that become evidence as a result of the teaching provided herein.

### Example 1: Preparation of Naloxone HCl 40 mg/mL Compositions

The use of nitrogen purging and effect of pH adjustment was studied on the four liquid compositions comprising Naloxone hydrochloride shown in Table 1.

TABLE 1

| | | | Quantity/mL | | | |
|---|---|---|---|---|---|---|
| | | | Comp. | Comp. | Comp. | Comp. |
| No. | Component | Function | 1 | 2 | 3 | 4 |
| 1 | Naloxone | Active | 40 mg | 40 mg | 40 mg | 40 mg |
| | Hydro- | | | | | |
| | chloride | | | | | |
| 2 | Disodium | Permeation | 2 mg | 2 mg | 2 mg | 2 mg |
| | edetate | enhancer | | | | |
| 3 | Sodium | Tonicity | 7 mg | 7 mg | 7 mg | 7 mg |
| | chloride | adjusting | | | | |
| | | agent | | | | |
| 4 | Hydrochloric | pH adjusting | — | — | q.s. to | — |
| | acid | agent | | | pH 3.5 | |
| 5 | Water | Solvent | q.s. to | q.s. to | q.s. to | q.s. to |
| | | | 1 mL | 1 mL | 1 mL | 1 mL |

| | | | | |
|---|---|---|---|---|
| Nitrogen purging to control | Yes | Yes | Yes | No |
| dissolved oxygen | | | | |
| Nitrogen purging to control | Yes | No | Yes | No |
| headspace oxygen | | | | |

Preparation: Partial quantity of water required for batch size (about 80% of batch size) was collected in suitable container and purged with nitrogen (wherever required in example). Required quantity of disodium edetate was added and mixed to dissolve. Required quantity of Naloxone hydrochloride was added and dissolved. Required quantity of sodium chloride was added mixed to dissolve. pH of the solution was adjusted using 0.1 N hydrochloric acid (if required). Volume was made up to batch size. The solution was filtered through a PVDF filter with pore size 0.22μ. Filtered solution was filled in 15 mL HDPE bottle keeping 10 mL fill volume and filled bottles were screw capped with spray pump with nitrogen purging in headspace (wherever required in example).

### Example 2: Stability Study

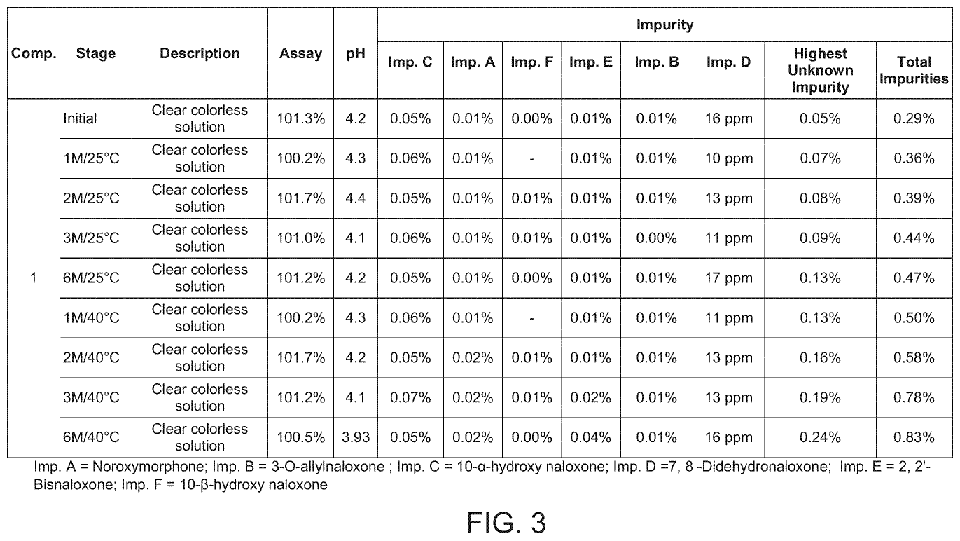

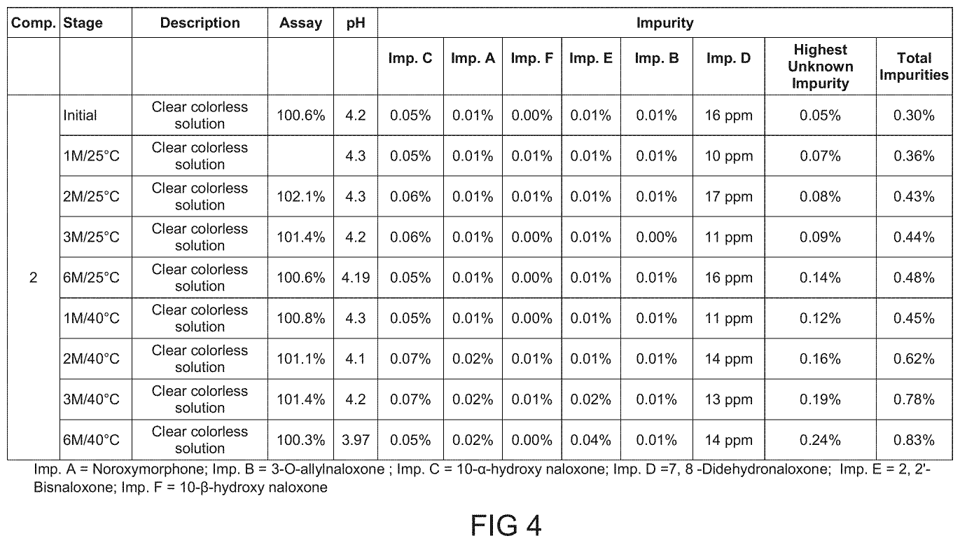

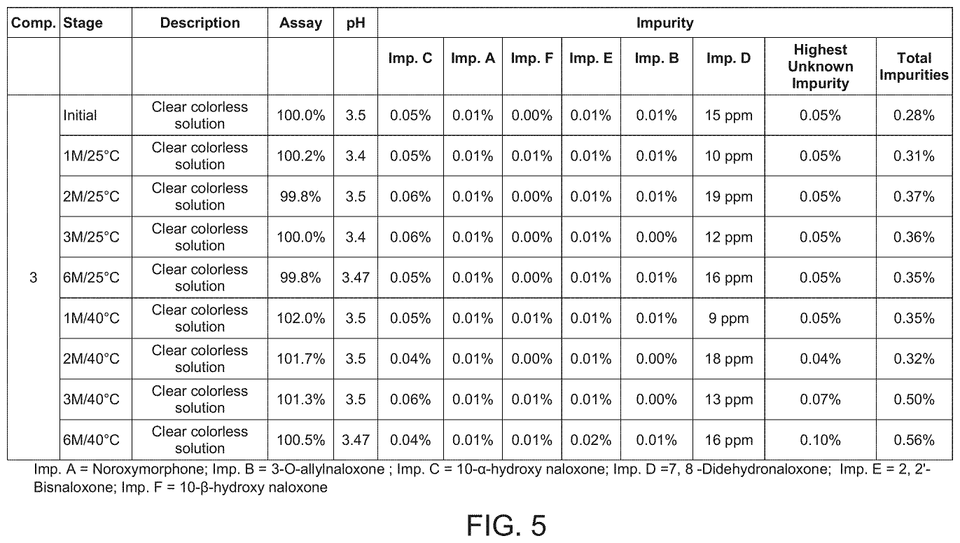

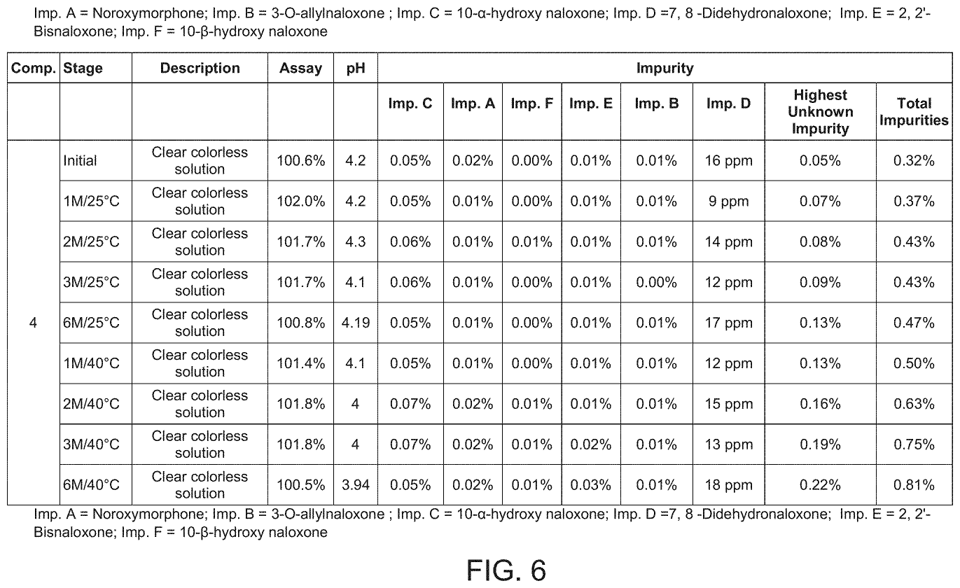

The stability of Compositions 1-4 of Example 1 was determined by placing the filled bottles capped with spray pump in controlled storage at 25° C. and 40° C. Samples were pulled at 1, 2, 3 and 6 months and analyzed for assay and impurities by HPLC. The pH was measured a using Mettler Toledo F20 pH meter. Results are shown in FIGS. 3-6.

### Example 3: Pump Delivery Study

A pump delivery study was performed using Composition 4 in Example 1. The dose in one spray was measured using one device after priming of 4 sprays. The amount of Naloxone at initial and after 6 months storage at 40° C. were determined by HPLC. The results are shown in Table 2.

TABLE 2
Target: 100.7 mg (100 μL) [Density = 1.007 gm/mL)

| | | | | After 6 | | After 6 |
|---|---|---|---|---|---|---|
| | Initial | | Initial | months/ | | months/ |
| Spray# | (mg) | | (%) | 40° C. (mg) | | 40° C. (%) |
| 1 | 98.20 | mg | 97.52% | 100.50 | mg | 99.80% |
| 2 | 99.30 | mg | 98.61% | 98.60 | mg | 97.91% |
| 3 | 100.30 | mg | 99.60% | 99.70 | mg | 99.01% |
| 4 | 98.80 | mg | 98.11% | 98.90 | mg | 98.21% |
| 5 | 97.40 | mg | 96.72% | 101.30 | mg | 100.60% |
| 6 | 103.30 | mg | 102.58% | 100.70 | mg | 100.00% |
| 7 | 96.60 | mg | 95.93% | 99.70 | mg | 99.01% |
| 8 | 96.80 | mg | 96.13% | 98.80 | mg | 98.11% |
| 9 | 98.10 | mg | 97.42% | 96.60 | mg | 95.93% |
| 10 | 103.40 | mg | 102.68% | 99.40 | mg | 98.71% |
| Minimum | 96.60 | mg | 95.93% | 96.60 | mg | 95.93% |
| Maximum | 103.40 | mg | 102.68% | 101.30 | mg | 100.60% |
| Mean | 99.22 | mg | 98.53% | 99.42 | mg | 98.73% |

| | | | | |
|---|---|---|---|---|
| Standard | 2.32 | 2.30 | 1.26 | 1.25 |
| Deviation | | | | |
| % RSD | 2.34 | 2.34 | 1.26 | 1.26 |

### Example 4: Naloxone HCl 40 mg/mL Composition

A 40 mg/mL Naloxone HCl composition with 10 mL per container is shown in Table 3.

TABLE 3

| Component | Function | Quantity/Unit | | % w/v |
|---|---|---|---|---|
| Naloxone | Active | 400 | mg | 4.0% |
| Hydrochloride | | | | |
| Sodium chloride | Tonicity | 10 | mg | 0.1% |
| | adjusting agent | | | |
| Disodium edetate | Permeation | 20 | mg | 0.2% |
| | enhancer | | | |

| | | | |
|---|---|---|---|
| Hydrochloric | pH adjusting | q.s. to pH | — |
| acid | agent | 4.0 to 5.0 | |
| Water | Solvent | q.s. to 10 mL | q.s. to 100% |

Preparation: Partial quantity of water required for batch size (about 80% of batch size) was collected in suitable container. Required quantity of disodium edetate was added and mixed to dissolve. Required quantity of Naloxone hydrochloride was added and dissolved. Required quantity of sodium chloride was added and mixed to dissolve. pH of the solution was adjusted using 0.1 N hydrochloric acid to between 4.0 and 5.0. Volume was made up to batch size. The solution was filtered through a suitable filter with pore size 0.2 to 5μ. Filtered solution was filled in HDPE bottle keeping 10 mL fill volume and filled bottles were screw capped with spray pump followed by cap.

### Example 5: Naloxone HCl 40 mg/mL Composition

An alternative composition of 40 mg/mL Naloxone HCl with 10 mL per container is shown in Table 4.

TABLE 4

| Component | Function | Quantity/Unit | | % w/v |
|---|---|---|---|---|
| Naloxone Hydrochloride | Active | 400 | mg | 4.0% |
| Phenylethyl alcohol | Preservative | 25 | mg | 0.25% |

| | | | |
|---|---|---|---|
| Hydrochloric acid | pH adjusting | q.s. to pH | — |
| | agent | 4.0 to 5.0 | |
| Water | Solvent | q.s. to 10 mL | q.s. to 100% |

Preparation: Partial quantity of water required for batch size (about 80% of batch size) was collected in suitable container. Required quantity of phenylethyl alcohol was added and mixed. Required quantity of Naloxone hydrochloride was added and dissolved. pH of the solution was adjusted using 0.1 N hydrochloric acid to between about 4.0 and 5.0. Volume was made up to batch size. The solution was filtered through a suitable filter with pore size 0.2 to 5μ. Filtered solution was filled in HDPE bottle keeping 10 mL fill volume and filled bottles were screw capped with spray pump followed by cap

### Example 6: Naloxone HCl 40 mg/mL Composition

An alternative composition of a 40 mg/mL Naloxone HCl with 10 mL per container is shown in Table 5.

TABLE 5

| Component | Function | Quantity/Unit | | % w/v |
|---|---|---|---|---|
| Naloxone Hydrochloride | Active | 400 | mg | 4.0% |
| Phenylethyl alcohol | Permeation | 25 | mg | 0.25% |
| | enhancer | | | |
| Disodium edetate | Chelating | 20 | mg | 0.2% |
| | agent | | | |

| | | | |
|---|---|---|---|
| Hydrochloric acid | pH adjusting | q.s. to pH | — |
| | agent | 4.0 to 5.0 | |
| Water | Solvent | q.s. to 10 mL | q.s. to 100% |

Preparation: Partial quantity of water required for batch size (about 80% of batch size) was collected in suitable container. Required quantity of disodium edetate and phenylethyl alcohol was added and mixed to get clear homogeneous solution. Required quantity of Naloxone hydrochloride was added and dissolved. pH of the solution was adjusted using 0.1 N hydrochloric acid to between 4.0 and 5.0. Volume was made up to batch size. The solution was filtered through a suitable filter with pore size 0.2 to 5μ. Filtered solution was filled in HDPE bottle keeping 10 mL fill volume and filled bottles were screw capped with spray pump followed by cap

### Example 7: Naloxone HCl 50 mg/mL Composition

A 50 mg/mL Naloxone HCl composition with 10 mL per container is shown in Table 6.

TABLE 6

| Component | Function | Quantity/Unit | % w/v |
|---|---|---|---|
| Naloxone Hydrochloride | Active | 500 mg | 5.0% |
| Hydrochloric acid | pH adjusting | q.s. to pH | — |
| | agent | 4.0 to 5.0 | |
| Water | Solvent | q.s. to 10 mL | q.s. to 100% |

Preparation: Partial quantity of water required for batch size (about 80% of batch size) was collected in suitable container. Required quantity of Naloxone hydrochloride was added and dissolved. pH of the solution was adjusted using 0.1 N hydrochloric acid to between 4.0 and 5.0. Volume was made up to batch size. The solution was filtered through a suitable filter with pore size 0.2 to 5μ. Filtered solution was filled in HDPE bottle keeping 10 mL fill volume and filled bottles were screw capped with spray pump followed by cap.

### Example 8

The composition of Example 5 was used but increasing bottle fill to 50 mL. The quantity per bottle is shown in Table 7.

TABLE 7

| Component | Function | Quantity/Unit | | % w/v |
|---|---|---|---|---|
| Naloxone Hydrochloride | Active | 2.0 | gm | 4.0% |
| Phenylethyl alcohol | Preservative | 125 | mg | 0.25% |

| | | | |
|---|---|---|---|
| Hydrochloric acid | pH adjusting | q.s. to pH | — |
| | agent | 4.0 to 5.0 | |
| Water | Solvent | q.s. to 50 mL | q.s. to 100% |

Preparation: Partial quantity of water required for batch size (about 80% of batch size) was collected in suitable container. Required quantity of phenylethyl alcohol was added and mixed to get clear homogenous solution. Required quantity of Naloxone hydrochloride was added and dissolved. pH of the solution was adjusted using 0.1 N hydrochloric acid to between 4.0 and 5.0. Volume was made up to batch size. The solution was filtered through a suitable filter with pore size 0.2 to 5μ. Filtered solution was filled in HDPE bottle keeping 50 mL fill volume and filled bottles were screw capped with spray pump followed by cap.

### Preparation: Example 9

Caffeine was added to composition of Example 8. The composition per bottle is shown in Table 8.

TABLE 8

| Component | Function | Quantity/Unit | | % w/v |
|---|---|---|---|---|
| Naloxone Hydrochloride | Active | 2.0 | gm | 4.0% |
| Phenylethyl alcohol | Preservative | 125 | mg | 0.25% |
| Caffeine | Permeation | 500 | mg | 1.0% |
| | enhancer | | | |

| | | | |
|---|---|---|---|
| Hydrochloric acid | pH adjusting | q.s. to pH | — |
| | agent | 3.0 to 6.0 | |
| Water | Solvent | q.s. to 50 mL | q.s. to 100% |

Preparation: Partial quantity of water required for batch size (about 80% of batch size) was collected in suitable container. Required quantity of phenylethyl alcohol and caffeine were added and mixed to get clear homogenous solution. Required quantity of Naloxone hydrochloride was added and dissolved. pH of the solution was adjusted using 0.1 N hydrochloric acid to between 3.0 and 6.0. Volume was made up to batch size. The solution was filtered through a suitable filter with pore size 0.2 to 5μ. Filtered solution was filled in HDPE bottle keeping 50 mL fill volume and filled bottles were screw capped with spray pump followed by cap.

Having now fully described this invention, it will be understood by those of ordinary skill in the art that the same can be performed within a wide and equivalent range of conditions, formulations, and other parameters without affecting the scope of the invention or any embodiment thereof.

What is claimed:

1. A multiple dose Naloxone spray dosage form, comprising:
a container;
ten or more unit doses of Naloxone or a pharmaceutically acceptable salt thereof in a liquid nasal carrier contained in the container;
a metered dosage spray pump and actuator provided with the container;
wherein each unit dose metered by the metered dosage spray pump contains about 50 μL to about 200 μL of Naloxone or a pharmaceutically acceptable salt thereof in a liquid nasal carrier; and
wherein each unit dose metered by the metered dosage spray pump contains about 0.5 mg to 12 mg of Naloxone or a pharmaceutically acceptable salt thereof.

2. The multiple dose Naloxone spray dosage form of claim 1, wherein the container contains 10 to 500 unit doses of Naloxone or a pharmaceutically acceptable salt thereof in a liquid nasal carrier.

3. The multiple dose Naloxone spray dosage form of claim 1, wherein the container contains about 1 to 50 mL of the Naloxone or a pharmaceutically acceptable salt thereof in a liquid nasal carrier.

4. The multiple dose Naloxone spray dosage form of claim 1, wherein a concentration of the Naloxone or pharmaceutically acceptable salt in the liquid nasal carrier is about 10 mg/m L to 60 mg/m L.

5. A multiple dose Naloxone spray dosage form, comprising:
a container;
two or more unit doses of Naloxone or a pharmaceutically acceptable salt thereof in a liquid nasal carrier contained in the container;
a metered dosage spray pump and actuator provided with the container.

6. The multiple dose Naloxone spray dosage form of claim 5, wherein the container contains 10 or more unit doses of Naloxone or a pharmaceutically acceptable salt thereof in a liquid nasal carrier.

7. The multiple dose Naloxone spray dosage form of claim 5, wherein the container contains 3 to 500 unit doses of Naloxone or a pharmaceutically acceptable salt thereof in a liquid nasal carrier.

8. The multiple dose Naloxone spray dosage form of claim 5, wherein the metered dosage spray pump delivers about 50 to about 200 μL of Naloxone or a pharmaceutically acceptable salt thereof in a liquid nasal carrier upon actuation by a user.

9. The multiple dose Naloxone spray dosage form of claim 8, wherein the metered dosage spray pump delivers about 100 μL of Naloxone or a pharmaceutically acceptable salt thereof in a liquid nasal carrier upon actuation by a user.

10. The multiple dose Naloxone spray dosage form of claim 7, wherein the container contains about 1 to 50 mL of the Naloxone or a pharmaceutically acceptable salt thereof in a liquid nasal carrier.

11. The multiple dose Naloxone spray dosage form of claim 5, wherein a concentration of the Naloxone or pharmaceutically acceptable salt in the liquid nasal carrier is about 10 mg/m L to 60 mg/m L.

12. The multiple dose Naloxone spray dosage form of claim 11, wherein each unit dose comprises 2 mg to 4 mg of Naloxone or a pharmaceutically acceptable salt thereof.

13. The multiple dose Naloxone spray dosage form of claim 5, wherein the intranasal carrier comprises a preservative.

14. The multiple dose Naloxone spray dosage form of claim 13, wherein the preservative is phenyl ethyl alcohol.

15. The multiple dose Naloxone spray dosage form of claim 5, wherein the intranasal carrier comprises permeation enhancer selected from disodium edetate and caffeine.

16. The multiple dose Naloxone spray dosage form of claim 5, wherein the intranasal carrier comprises acid sufficient to adjust pH to about 3 to about 6.

17. A method of treating opioid overdose or toxicity in a patient by intranasally administering to the patient multiple doses of Naloxone in a liquid carrier contained in a multiple dose spray dispenser containing two or more unit doses of the Naloxone in a liquid carrier.

18. The method of claim 17, wherein:
the multiple dose spray dispenser is placed in a first nostril of the patient, and the spray dispenser is actuated to spray the Naloxone in a liquid carrier in the first nostril;
the multiple dose spray dispenser is placed in a second nostril of the patient, and the spray dispenser is actuated to spray the Naloxone in a liquid carrier in the second nostril.

19. The method of claim 18, further comprising:
repeating the steps of claim 18 procedure using same multiple dose spray dispenser every two to three minutes as needed.

20. The method of claim 17, wherein each dose contains about 0.5 mg to 6 mg of Naloxone or a pharmaceutically acceptable salt thereof.
